(19) United States
(12) Patent Application Publication
(10) Pub. No.: US 2018/0333514 A1
(43) Pub. Date: Nov. 22, 2018

(54) DISPOSABLE ABSORBENT ARTICLE INCLUDING AN ELASTIC HOT MELT ADHESIVE BACK SHEET

(71) Applicant: H.B. Fuller Company, St. Paul, MN (US)

(72) Inventors: Tara Misra, Shoreview, MN (US); Sehban N. Ozair, Blaine, MN (US); Kevin P. Davis, Woodbury, MN (US); Ameara S. Mansour, Woodbury, MN (US)

(21) Appl. No.: 15/982,692

(22) Filed: May 17, 2018

Related U.S. Application Data
(60) Provisional application No. 62/507,579, filed on May 17, 2017.

(51) Int. Cl. A61L 15/42; A61F 13/15; A61F 13/514; A61F 13/515; A61L 15/24; A61L 15/58

(52) U.S. Cl. CPC A61L 15/42; A61F 13/15739; A61F 13/51401; A61F 2013/5147; A61F 13/515; A61L 15/24; A61L 15/58; A61F 13/51464

(57) ABSTRACT

The invention features a disposable absorbent article including a top sheet, an elastic hot melt adhesive back sheet, and a positioning adhesive, the top sheet and the elastic hot melt adhesive back sheet, being adhered to each other to form a layered assembly, the positioning adhesive being located on the garment facing surface of the elastic hot melt adhesive back sheet, the elastic hot melt adhesive including from about 30% by weight to about 80% by weight of a thermoplastic polymer and a tackifying agent.

This application claims the benefit of U.S. Provisional Application No. 62/507,579 filed May 17, 2017, which is incorporated herein.

### BACKGROUND

In the area of industrial adhesives, hot melt adhesives are commonly used to bond together a wide variety of articles including disposable absorbent articles comprising non-woven substrates e.g. adult incontinence products (e.g. briefs and pads), disposable diapers, feminine hygiene articles (e.g. sanitary napkins, panty liners, etc.), bed pads, puppy pads, medical dressings, etc.

There are adhesives used in the manufacture of a feminine hygiene articles and adult incontinence pads. For example, adhesives can be used for construction (e.g. bonding the back sheet to the top sheet and optionally the absorbent core), for core stabilization (e.g. applying an adhesive to the absorbent core to increase the strength of the core) and for positioning (e.g. the adhesive on the back of the sanitary napkin or panty liner that holds the article to the user's undergarment).

Modern day feminine hygiene and adult incontinence articles are fairly effective. However, there can still be issues with fit, especially when the user is active or wishes to use the article with different styles of undergarments.

There is a need in the industry for a simple, effective way of improving the fit and function of feminine hygiene and adult incontinence articles.

### SUMMARY

In one aspect, the invention features a disposable absorbent article including a top sheet, an elastic hot melt adhesive back sheet, and a positioning adhesive, the top sheet and the elastic hot melt adhesive back sheet, being adhered to each other to form a layered assembly, the positioning adhesive being located on the garment facing surface of the elastic hot melt adhesive back sheet, the elastic hot melt adhesive comprising from about 30% by weight to about 80% by weight of a thermoplastic polymer and a tackifying agent.

In one embodiment, the elastic hot melt adhesive has a Mechanical Hysteresis as tested by the Mechanical Hysteresis test method (Test Lamination Preparation 1, 60 gsm, 1st cycle) of from about 0.1 to about 0.8. In a different embodiment, the thermoplastic polymer is a styrene block copolymer. In another embodiment, the thermoplastic polymer is present at from about 40% by weight to about 70% by weight and the tackifying agent is present at from about 5% by weight to about 40% by weight.

In a different embodiment, the elastic hot melt adhesive has a Brookfield Viscosity at 177° C. of from about 500 cP to about 30,000 cP. In another embodiment, the elastic hot melt adhesive back sheet is liquid impermeable. In a second embodiment, the hot melt adhesive back sheet has a thickness of from about 5 gsm to about 75 gsm.

In one embodiment, the article is free of an absorbent core. In another embodiment, the top sheet is selected from the group consisting of a high loft top sheet and a top sheet with multiple layers. In another embodiment, the top sheet is stretchable.

In a different embodiment, the disposable absorbent article is selected from the group consisting of a panty liner, a sanitary napkin and an incontinence pad. In another embodiment, the positioning adhesive is a hot melt pressure sensitive adhesive. In one embodiment, the positioning adhesive is applied in a comb pattern.

In another aspect, the invention features a method of forming a disposable absorbent article including the steps of applying a molten elastic hot melt adhesive in a full coat layer to the garment facing side of a top sheet, and applying a positioning adhesive to the garment facing side of the elastic hot melt adhesive.

In one embodiment, a disposable article made by the method. In another embodiment, the disposable article is selected from the group consisting of a panty liner, a sanitary napkin and an incontinence pad. In a different embodiment, the molten elastic hot melt adhesive is applied to the top sheet by a non-contact slot die application method.

This invention includes a simple, disposable absorbent article including an elastic hot melt adhesive back sheet that can move with the user and/or be stretched and attached to the garment to accommodate different sizes of under garments.

### Definitions

Stretchable as used herein means that the layer referred to deforms when subjected to an external load while maintaining its functionality. Stretchable as applied to the disposable article means that the article can stretch at least 10% when subjected to a 10 Newton (N) tensile load while still maintaining its functionality.

Elastic as used herein means that the layer referred to deforms when subjected to an external load and recovers, at least partially, from the deformation when the load is removed.

### DETAILED DESCRIPTION

### Disposable Absorbent Article

The invention features a disposable absorbent article including a top sheet, an elastic hot melt adhesive back sheet, and a positioning adhesive, the top sheet and the elastic hot melt adhesive back sheet, being adhered to each other to form a layered assembly, the positioning adhesive being located on the garment facing surface of the elastic hot melt adhesive back sheet, the elastic hot melt adhesive including from about 30% by weight to about 80% by weight of a thermoplastic polymer and a tackifying agent.

The properties required can vary according to the demands of the disposable absorbent article. For some applications, the disposable absorbent article needs only enough elasticity to remain in place as the user is active. In other applications, the disposable absorbent article needs to be stretchable to enable the user to stretch the disposable absorbent article into place and adhere it into the garment.

The disposable absorbent article can be selected from the group consisting of a panty liner, a sanitary napkin, an incontinence pad, a bed pad, a puppy pad and a wound care article (e.g. medical dressing).

### Top Sheet

The top sheet is liquid-permeable and makes up the body facing layer of the disposable absorbent article. The top sheet can be stretchable (i.e. extensible). Post treatments can be used to make it extensible.

The top sheet may be constructed from a wide range of suitable materials including apertured films, nonwoven webs of natural fibers (e.g., wood or cotton) or synthetic fibers (e.g., polypropylene or polyester) or a combination of such fibers. The top sheet can include high loft materials or multiple layers of materials. High loft is a general term for low density, thick or bulky materials, as compared to flat, paper like materials. It is characterized by a relatively high ratio of thickness to weight per unit area.

In one embodiment, the disposable absorbent article is free of an absorbent core and the top sheet alone provides absorbency to the disposable absorbent article.

The top sheet can be hydrophobic or hydrophilic (e.g. surfactant treated). The top sheet can be hydrophobic, with the exception of the area in the center which can be treated with surfactant to be hydrophilic.

### Elastic Hot Melt Adhesive Back Sheet

The back sheet is an elastic hot melt adhesive. It is adhered to the top sheet. The elastic hot melt adhesive can be tack-free once cooled to room temperature. The elastic hot melt adhesive back sheet is liquid-impermeable and makes up the garment facing layer of the disposable absorbent article. The elastic hot melt adhesive is present as a full coat film with a thickness of from about 5 grams per square meter (gsm) to about 75 gsm, from about 8 gsm to about 60 gsm, or even from about 10 gsm to about 50 gsm.

The elastic hot melt adhesive has a viscosity at 177° C. of from about 100 centipoise (cP) to about 50,000 cP, from about 500 cP to about 30,000 cP, or even from about 1,000 cP to about 15,000 cP. The elastic hot melt adhesive can be of a low enough viscosity to enable application with standard hot melt application equipment e.g. gears pump, slot die applicator, etc.

The elastic hot melt adhesive has elasticity as witnessed by a Mechanical Hysteresis as tested by the Mechanical Hysteresis test method (Test Lamination Preparation 1, 60 gsm, 1st cycle) of from about 0.1 to about 0.8, or even from about 0.2 to about 0.6.

The elastic hot melt adhesive includes a thermoplastic polymer and optionally a tackifying agent.

Thermoplastic Polymer

The elastic hot melt adhesive includes a thermoplastic polymer. The thermoplastic polymer is elastic. The thermoplastic polymer can be selected from the group consisting of styrene block copolymers and olefin polymers.

The thermoplastic polymer can be present at from about 30% by weight to about 80% by weight, from about 40% by weight to about 70% by weight, or even from about 45% by weight to about 70% by weight.

Styrene block copolymers include an aromatic vinyl polymer block and a conjugated diene polymer block, a hydrogenated conjugated diene polymer block, or a combination thereof. The blocks can be arranged in a variety of configurations including, e.g., linear, branched, radial, star block, and combinations thereof. The aromatic vinyl polymer block can be derived from a variety of aromatic vinyl compounds including, e.g., styrene, alpha-methylstyrene, beta-methylstyrene, o-, m-, p-methylstyrene, t-butylstyrene, 2,4,6-trimethylstyrene, monofluorostyrene, difluorostyrene, monochlorostyrene, dichlorostyrene, methoxystyrene, 1,3-vinylnaphthalene, vinylanthracene, indene, acenaphthylene, and combinations thereof. The diene polymer block can be derived from a variety of diene-containing compounds including, e.g., isoprene, butadiene, hexadiene, 2,3-dimethyl-1,3-butadiene, 1,3-pentadiene, and hydrogenated versions thereof, and combinations thereof.

Useful styrene block copolymers include, e.g., diblock, triblock and multiblock copolymers including, e.g., styrene-butadiene, styrene-butadiene-styrene, styrene-isoprene, styrene-isoprene-styrene, styrene-ethylene/butene, styrene-ethylene/butene-styrene, styrene-ethylene/propylene, styrene-ethylene/propylene-styrene, styrene-ethylene-ethylene/propylene-styrene, farnesene styrene and combinations thereof and hydrogenated or functionalized versions thereof.

Useful styrene block copolymers include VECTOR 6241 (Linear, styrene-butadiene-styrene, pure triblock copolymer, 43 weight % styrene, Melt Flow Rate (MFR) (200° C./5 kilogram)=23 decigram (dg)/minute (min)), VECTOR 8508 (Linear, styrene-butadiene-styrene, pure triblock copolymer, 29 weight % styrene, MFR (200° C./5 kg)=12 dg/min and VECTOR 4411 (Linear, styrene-isoprene-styrene, pure triblock copolymer, 44 weight % styrene, MFR (200° C./5 kg)=40 dg/min) all available from TSRC Dexco (Houston, Tex.).

The thermoplastic polymer can be an olefin polymer. Useful olefin polymers include propylene and polyethylene based homopolymers and copolymers and olefin block copolymers. The olefin polymer can be a single-site catalyzed (e.g. metallocene catalyzed) polymer.

Useful commercially available olefin polymers include propylene ethylene copolymers e.g. VISTAMAXX 6202 and VISTAMAXX 6102 available from ExxonMobil Chemical (Houston, Tex.) and propylene ethylene copolymer, VERSIFY 3000 and olefin block copolymers, INFUSE 9507 and INFUSE 9807 and available from Dow Chemical Company (Houston, Tex.).

The thermoplastic polymer can include one or more thermoplastic polymers.

Tackifying Agent

The elastic hot melt adhesive can include a tackifying agent. The tackifying agent can be fluid or solid at room temperature. Suitable classes of tackifying agents include, e.g., aromatic, aliphatic and cycloaliphatic hydrocarbon resins, mixed aromatic and aliphatic modified hydrocarbon resins, aromatic modified aliphatic hydrocarbon resins, and hydrogenated versions thereof; terpenes, modified terpenes and hydrogenated versions thereof; natural rosins, modified rosins, rosin esters, and hydrogenated versions thereof; low molecular weight polylactic acid; and combinations thereof. Examples of useful natural and modified rosins include gum rosin, wood rosin, tall oil rosin, distilled rosin, hydrogenated rosin, dimerized rosin and polymerized rosin. Examples of useful rosin esters include e.g., glycerol esters of pale wood rosin, glycerol esters of hydrogenated rosin, glycerol esters of polymerized rosin, pentaerythritol esters of natural and modified rosins including pentaerythritol esters of pale wood rosin, pentaerythritol esters of hydrogenated rosin, pentaerythritol esters of tall oil rosin, and phenolic-modified pentaerythritol esters of rosin. Useful tackifying agents are commercially available under a variety of trade designations including, e.g., hydrocarbon resins under the ESCOREZ series of trade designations from Exxon Mobil Chemical Company (Houston, Tex.), including ESCOREZ 5400 (1% aromatic content), ESCOREZ 5415, ESCOREZ 5600 (9.8% aromatic content), ESCOREZ 5690 (10% aromatic content), ESCOREZ 5615 (9.9% aromatic content), aliphatic hydrocarbon resins under the EASTOTAC series of trade designations from Eastman Chemical (Kingsport, Tenn.) including EASTOTAC H-100R and EASTOTAC H-100L, and hydrocarbon resins available under the WINGTACK series of trade designations from Cray Valley HSC (Exton, Pa.) including WINGTACK 86, WINGTACK EXTRA, and WINGTACK 95 and aliphatic hydrocarbon resins under the PICCOTAC and aromatic hydrocarbon resins under the KRISTALEX and PLASTOLYN series of trade designations from Eastman Chemical Company (Kingsport, Tenn.) including, e.g., PICCOTAC 8095, KRISTALEX 3100, PLASTOLYN 240 and PLASTOLYN 290.

The adhesive composition can include at least one tackifying agent with aromatic content. The tackifying agent can have an aromatic content of greater than 5%, greater than 20%, greater than 50%, from about 5% to about 20% by weight, from about 7.5% to about 15% by weight, or even from 5% by weight to less than 10% by weight. The aromatic content is measured by Nuclear Magnetic Resonance (NMR) spectroscopy.

The composition can include a tackifying agent with a softening point of less than about 97° C., or even from about 70° C. to about 95° C.

The elastic hot melt adhesive can include from about 5% to about 40% by weight, from about 10% to about 35% by weight, or even from about 10% to about 30% by weight of a tackifying agent.

Plasticizer

The elastic hot melt adhesive can further include a plasticizer. The plasticizer can be saturated (e.g. mineral oil, paraffinic oil, etc.) in order to improve heat stability for bulk handling. Suitable plasticizers include, e.g., naphthenic oils, paraffinic oils (e.g., cycloparaffin oils), mineral oils, phthalate esters, adipate esters, olefin oligomers (e.g., oligomers of polypropylene, polybutene, and hydrogenated polyisoprene), polybutenes, polyisoprene, hydrogenated polyisoprene, polybutadiene, benzoate esters, animal oil, plant oils (e.g. castor oil, soybean oil), derivatives of oils, glycerol esters of fatty acids, polyesters, polyethers, lactic acid derivatives and combinations thereof.

Additional Materials

The elastic hot melt adhesive can also include additional materials such as waxes, stabilizers, antioxidants, additional thermoplastic polymers (e.g. amorphous poly-alpha olefins), adhesion promoters, ultraviolet light stabilizers, corrosion inhibitors, colorants (e.g., pigments and dyes), fillers, surfactants, wetness indicators, superabsorbents, odor reducing materials and combinations thereof.

### Absorbent Core

The disposable absorbent article can include an absorbent core. The absorbent core includes the core and optionally a core wrap. The core can vary in design, shape, and materials used. It can be shaped like a long rectangle or like a dog bone. The core is generally centered within the article, and is firmly secured between the top sheet and elastic hot melt adhesive back sheet. The core includes absorbent materials able to absorb bodily fluids and solids received through the top sheet. Absorbent materials can include fluff (e.g. cellulose based), super absorbent (e.g. super absorbent particles), nonwoven, tissue, foam, or any other absorbent material.

The core wrap can be any material that wraps around the core to hold the components of the core in place. The core wrap generally wraps around the two longer sides of the core, leaving the ends open. The core wrap can be selected from the group consisting of nonwoven and tissue.

### Acquisition Layer

The disposable absorbent article can include an acquisition layer. The acquisition layer is a layer of material between the core and the top sheet and assists in directing the bodily fluids and solids into the core. The acquisition layer can be a hydrophilic treated nonwoven or an apertured film.

### Positioning Adhesive

The disposable absorbent article includes a positioning adhesive to keep it in place when located in the garment. The positioning adhesive can be a pressure sensitive hot melt adhesive. The positioning adhesive can include a styrene block copolymer.

### Method of Forming a Disposable Absorbent Product

The invention includes a method of forming a disposable absorbent article including applying a molten elastic hot melt adhesive in a full coat layer to the garment facing side of a top sheet, and applying a positioning adhesive to the garment facing side of the elastic hot melt adhesive.

The application method to apply the elastic hot melt adhesive is not particularly limited. Any application method can be used which results in a full coat layer i.e. a layer with 100% elastic hot melt adhesive coverage. The elastic hot melt adhesive can be applied as a pre-coated film. The elastic hot melt adhesive can be applied in a molten state by non-contact or by contact application at an application temperature of from about 125° C. to about 190° C. The elastic hot melt adhesive can be applied with a slot applicator. One layer of elastic hot melt adhesive can be used, alternatively two layers can be used one on top of the other. The applicator can be fed by either a gear pump, an extruder or a constant pressure pump.

The positioning adhesive is a pressure sensitive adhesive and is preferably a hot melt adhesive. It can be applied in a molten state using a variety of patterns depending on the effect desired. In one embodiment, it is applied with a comb pattern to get “stripes” of adhesive that run perpendicular to the length of the disposable absorbent article (i.e. cross direction). The “stripes” of adhesive can also run parallel to the length of the disposable absorbent article. The positioning adhesive can be applied directly to the elastic hot melt adhesive. Alternatively, the positioning adhesive can be applied to a release liner and then transfer coated to the garment facing side of the elastic hot melt adhesive.

The invention will now be described by way of the following examples. All parts, ratios, percents and amounts stated in the Examples are by weight unless otherwise specified.

### EXAMPLES

Test Methods

Test procedures used in the examples and throughout the specification, unless stated otherwise, include the following.

Viscosity Test Method

Viscosity is determined in accordance with ASTM D-3236 entitled, “Standard Test Method for Apparent viscosity of Adhesives and Coating Materials,” (Oct. 31, 1988), using a Brookfield Thermosel viscometer Model RVDV 2 and a number 27 spindle. The results are reported in centipoise (cP).

Test Lamination Preparation 1

Test laminations for examples 1 and 2 were prepared using a Nordson Trio continuous slot die applicator. The elastic hot melt adhesive composition was applied through the nonwoven facing die while the positioning adhesive was applied through the free side facing die in a comb pattern. These layers were directly coated to a nonwoven* substrate and combined with a release liner at the coater nip. The adhesives were applied at an application temperature of 160° C., a nip pressure of 1.05 Newton/millimeter (N/mm) (6 pounds per linear inch (PLI)), and a run speed of at least 25 meters (m)/minute (min) (82 feet (ft)/min). *The nonwoven used is carded, hydroentangled and comprises 50/50 (PET/PP). It has a basis weight of 30 gsm and a cross direction tensile strength of 18 Newton (N)/5 cm.

Laminations were prepared with an elastic hot melt adhesive coat weight of 40 and 60 gsm and the width of the adhesive was at least 8.9 centimeters (cm) (3.5 inches (in)), and an overall positioning adhesive coat weight of 20 gsm in an overall width of at least 8.9 centimeters (cm) (3.5 in) coated on top of the elastic hot melt adhesive layer in a comb pattern using the Nordson TRIO applicator. The comb shim pattern consisted of alternating stripes of positioning adhesive application and voids where no adhesive was applied. Positioning adhesive stripes were 4 mm wide and the void spaces were 3 mm wide, this pattern was repeated for a total coating width of 8.9 cm (3.5 in). A sufficient amount of laminate is prepared such that at least 1.5 m (60 in) of representative lamination can be collected for testing.

Test Lamination Preparation 2

Test laminations for examples 3 and 4 were prepared using two continuous slot die applicators. The thermoplastic adhesive composition was applied through one slot die applicator onto the nonwoven* substrate, while the positioning adhesive was applied through the second applicator in a comb pattern onto a release liner. These laminations were then combined at the nip. The adhesives were applied at an application temperature of 160° C., a nip pressure of 1.05 Newton/millimeter (N/mm) (6 pounds per linear inch (PLI)), and a run speed of at least 25 meters (m)/minute (min) (82 feet (ft)/min). *The nonwoven used is carded, hydroentangled and comprises 50/50 (PET/PP). It has a basis weight of 30 gsm and a cross direction tensile strength of 18 Newton (N)/5 cm.

Laminations were prepared with an elastic hot melt adhesive coat weight of 40 and 60 gsm and the width of the adhesive was at least 8.9 centimeters (cm) (3.5 inches (in)), and an overall positioning adhesive coat weight of 20 gsm in an overall width of at least 8.9 centimeters (cm) (3.5 in). The hot melt positioning adhesive was applied using a second slot die applicator onto a release liner and transfer coated to the back side of the elastic hot melt adhesive in a cross direction comb pattern with 20 gsm in the adhesive regions. The comb shim pattern is as described in Test Lamination Preparation 1. A sufficient amount of laminate is prepared such that at least 1.5 m (60 in) of representative lamination can be collected for testing.

Mechanical Hysteresis

Test laminates were prepared according to the Test Lamination Preparation Test 1 or 2 methods. Test samples are prepared by cutting the laminates in cross-web direction, with 2.54 cm (1 in) in width and at least 8.9 cm (3.5 in) in length. Grip separation is set to 5.1 cm (2 in). The test is conducted at least 24 hours after the non-woven laminate is prepared.

The strips prepared are extended to a 50% strain and then retracted to their original dimension. Subsequently the specimen goes through a second extension-retraction cycle with the same deformation. The cross head speed is set to 50.8 cm/min (20 in/min) during the extension portion of the cycle and 101.6 cm/min (40 in/min) during the retraction portion of the cycle. There is no holding time between extension and retraction. The samples are run on an INSTRON type-test instrument with at least 3 replicates. The energy loss during each hysteresis cycle and the total hysteresis cycle energy are recorded. The mechanical hysteresis is the ratio of the energy loss during the hysteresis cycle to the total hysteresis energy. Load at 50% extension (N) is also recorded.

TABLE ONE

| | Example | Example | Example | Example |
|---|---|---|---|---|
| | 1 | 2 | 3 | 4 |
| Test | 1 | 1 | 2 | 2 |
| Lamination | | | | |
| Preparation | | | | |
| Method | | | | |
| Elastic hot | CON- | CON- | CON- | CON- |
| melt adhesive | FORMA | FORMA | FORMA | FORMA |
| | 9800 | 9800 | 9800 | 9800 |
| Elastic hot melt | 60 | 40 | 60 | 40 |
| adhesive coat | | | | |
| weight (gsm) | | | | |
| Positioning | NW1043 | NW1043 | NW1043 | NW1043 |
| adhesive | | | | |
| Mechanical | | | | |
| Hysteresis | | | | |
| 1^{st} cycle | 0.473 | 0.584 | 0.626 | 0.669 |
| 2^{nd} cycle | 0.338 | 0.446 | 0.373 | 0.421 |
| Load at 50% | | | | |
| extension | | | | |
| (Newtons) | | | | |
| 1^{st} cycle | 1.14 | 0.74 | 3.41 | 2.13 |
| 2^{nd} cycle | 1.03 | 0.66 | 2.77 | 1.66 |

CONFORMA 9800 AND NW1043 are both available from H.B. Fuller Company.
CONFORMA 9800 has a viscosity at 177° C. (350° F.) of 7000 cP.

Other embodiments are within the claims.

What is claimed is:

1. A disposable absorbent article, comprising:
a. a top sheet,
b. an elastic hot melt adhesive back sheet, and
c. a positioning adhesive,
the top sheet and the elastic hot melt adhesive back sheet, being adhered to each other to form a layered assembly, the positioning adhesive being located on the garment facing surface of the elastic hot melt adhesive back sheet,
the elastic hot melt adhesive comprising from about 30% by weight to about 80% by weight of a thermoplastic polymer and a tackifying agent.

2. The disposable absorbent article of claim 1 wherein the elastic hot melt adhesive has a Mechanical Hysteresis as tested by the Mechanical Hysteresis test method (Test Lamination Preparation 1, 60 gsm, 1^{st} cycle) of from about 0.1 to about 0.8.

3. The disposable absorbent article of claim 1 wherein the thermoplastic polymer is a styrene block copolymer.

4. The disposable absorbent article of claim 1 wherein the thermoplastic polymer is present at from about 40% by weight to about 70% by weight and the tackifying agent is present at from about 5% by weight to about 40% by weight.

5. The disposable absorbent article of claim 1 wherein the elastic hot melt adhesive has a Brookfield Viscosity at 177° C. of from about 500 cP to about 30,000 cP.

6. The disposable absorbent article of claim 1 wherein the elastic hot melt adhesive back sheet is liquid impermeable.

7. The disposable absorbent article of claim 1 wherein the hot melt adhesive back sheet has a thickness of from about 5 gsm to about 75 gsm.

8. The disposable absorbent article of claim 1 wherein the disposable absorbent article is free of an absorbent core.

9. The disposable absorbent article of claim 1 wherein the top sheet is selected from the group consisting of a high loft top sheet and a top sheet with multiple layers.

10. The disposable absorbent article of claim 1 wherein the top sheet is stretchable.

11. The disposable absorbent article of claim 1 selected from the group consisting of a panty liner, a sanitary napkin and an incontinence pad.

12. The disposable absorbent article of claim 1 wherein the positioning adhesive is a hot melt pressure sensitive adhesive.

13. The absorbent article of claim 1 wherein the positioning adhesive is applied in a comb pattern.

14. A method of forming a disposable absorbent article comprising the steps of:
a. applying a molten elastic hot melt adhesive in a full coat layer to the garment facing side of a top sheet, and
b. applying a positioning adhesive to the garment facing side of the elastic hot melt adhesive.

15. A disposable article made by the method of claim 14.

16. The disposable article of claim 15 selected from the group consisting of a panty liner, a sanitary napkin and an incontinence pad.

17. The method of claim 15 wherein the molten elastic hot melt adhesive is applied to the top sheet by a non-contact slot die application method.
